(19) United States
(12) Patent Application Publication
(10) Pub. No.: US 2016/0143820 A1
(43) Pub. Date: May 26, 2016

(54) COLORFUL GEL AND PREPARATION METHOD THEREOF

(71) Applicant: Guangzhou Sheencolor Cosmetics Co., Ltd., Guangzhou (CN)

(72) Inventors: Weijian Deng, Guangzhou (CN); Ping Luo, Guangzhou (CN)

(21) Appl. No.: 14/798,369

(22) Filed: Jul. 13, 2015

(30) Foreign Application Priority Data
Nov. 24, 2014 (CN) 2014106827111

(51) Int. Cl. A61K 8/04; A61Q 1/02

(52) U.S. Cl. CPC A61K 8/042; A61Q 1/02

(57) ABSTRACT

The present invention provides a colorful gel, comprising following components by weight percentage: 20.0% to 25.0% by weight of glycerin, 20.0% to 25.0% by weight of propylene glycol, 1.0% by weight of sea algae extract, 0.05% by weight of disodium EDTA, 0.06% by weight of allantoin, 0.05% by weight of sodium hyaluronate, 20.0% to 30.0% by weight of cyclopentasiloxane, 5.0 to 10.0% by weight of dimethicone, 0.5 to 1.0% by weight of sodium acrylate/sodium acryloyldimethyl taurate copolymer, 0.25 to 0.5% by weight of isohexadecane, 0.2% to 0.4% by weight of polysorbate-80, 1.5% by weight of soluble collagen, 0.5% by weight of caprylyl glycol, and water as the rest. The present invention also provides a preparation method of the colorful gel. The colorful gel of the present invention has excellent moisturizing performance, and it can keep moisturizing skin, which is safe and healthy.

### TECHNICAL FIELD

The present invention relates to the field of cosmetic, and particularly a colorful skin care gel, and preparation method of this colorful skin care gel.

### BACKGROUND OF THE INVENTION

There are mainly two types of transparent gel sold in the market currently, one of them is aqueous gel, it is prepared by taking water-soluble polymer materials as a thickening agent, in combination of various water-soluble components; and the other one is oily gel, which takes oily raw materials as main components. These two types of gels have following defects: (1) only water-soluble raw materials can be added for the aqueous gel, and only oily components can be added for the oily gel, which restricts the selection of formula components considerably; (2) only water-soluble components can be added for the aqueous gel, so the nourishing performance is poor, and since only oily components can be added for the oily gel, it has problems such as sticky skin feel, or clogged pores; (3) in general, it is necessary to add preservative to prevent product from being contaminated; (4) it is necessary to add organic pigment or inorganic pigment to achieve color effect.

### SUMMARY OF THE INVENTION

It is an object of the present invention to provide a kind of transparent skin care gel with colorful appearance, good moisturizing performance, high content of oily component, and no preservative, with regarding to above technical problems.

It is a further object of the present invention to provide a preparation method of the colorful gel described above.

In order to achieve above objects, the present invention provides a colorful gel, comprising following components by weight percent:

20.0% to 25.0% by weight of glycerin,

20.0% to 25.0% by weight of propylene glycol,

1.0% by weight of sea algae extract,

0.05% by weight of disodium EDTA,

0.06% by weight of allantoin,

0.05% by weight of sodium hyaluronate,

20.0% to 30.0% by weight of cyclopentasiloxane,

5.0% to 10.0% by weight of dimethicone,

0.5% to 1.0% by weight of sodium acrylate/sodium acryloyldimethyl taurate copolymer,

0.25% to 0.5% by weight of isohexadecane,

0.2% to 0.4% by weight of polysorbate-80,

1.5% by weight of soluble collagen,

0.5% by weight of caprylyl glycol, and water as the rest.

The present invention also provides a method for preparing the colorful gel, comprising the steps of:

(1) wetting sodium hyaluronate with propylene glycol, then adding them into an aqueous phase pot and stirring them, adding deionized water, glycerin, sea algae extract, disodium EDTA, and allantoin successively to form an aqueous phase pot mixture, then heating the mixture to 80° C., and keeping the temperature constant for 10 minutes;

(2) adding dimethicone and part of cyclopentasiloxane successively into an emulsifying pot, stirring and mixing them thoroughly, and then heating them to 70° C.;

(3) mixing remaining cyclopentasiloxane, sodium acrylate/sodium acryloyldimethyl taurate copolymer, isohexadecane and polysorbate-80 thoroughly, then adding them into the emulsifying pot, and stirring them until homogenous;

(4) operating the emulsifying pot at a stirring speed of 300 revolutions per minute, and absorbing the mixture prepared in step (1) by vacuum suction, maintaining vacuum during reaction, after homogenizing for 5 minutes, obtaining an emulsifying pot mixture;

(5) cooling the emulsifying pot mixture to below 45° C. by stirring, adding caprylyl glycol and soluble collagen to get a final mixture, after thorough stirring and homogenizing, cooling the final mixture to 38 and discharging it.

Preferably, the cyclopentasiloxane used in step (2) accounts for 95˜97% of all the cyclopentasiloxane by weight.

Compared with prior art, the colorful skin care gel of the present invention has overcome defects of traditional gel, it applies advanced gel formula system, and it is beautiful and fashionable due to its lavender color by adjusting refractive indexes of aqueous phase and oil phase; the present invention comprises a great amount of moisturizing grease, making the combination of both long-acting and short-acting double-effect ultra moisturizing components possible, and has an excellent moisturizing performance (moisturizing effect can be kept for 24 hours), and the water content of skin cuticle will be increased significantly. Moreover, the present invention applies special thickening and emulsifying system, the obtained product has smooth texture and silky skin feel, and it is without preservatives, safe and environmentally friendly.

### DETAILED DESCRIPTION OF THE INVENTION

The invention is further illustrated by the following examples without limiting the scope of the invention to these particular embodiments.

All components are taken according to following weight percents, and all following components are commercially available.

| Group | Component | Example 1 | Example 2 | Example 3 |
|---|---|---|---|---|
| A | deionized water | To 100 | To 100 | To 100 |
| | sodium hyaluronate | 0.05 | 0.05 | 0.05 |
| | propylene glycol | 25.0 | 20.0 | 20.0 |
| | glycerin | 20.0 | 25.0 | 25.0 |
| | sea algae extract | 1.0 | 1.0 | 1.0 |
| | disodium EDTA | 0.05 | 0.05 | 0.05 |
| | allantoin | 0.06 | 0.06 | 0.06 |
| B | dimethicone | 5.0 | 10.0 | 5.0 |
| | cyclopentasiloxane | 25.0 | 19.0 | 29.0 |
| C | cyclopentasiloxane | 1.0 | 1.0 | 1.0 |
| | sodium acrylate/sodium | 0.5 | 0.8 | 1.0 |
| | acryloyldimethyl taurate | | | |
| | copolymer | | | |
| | isohexadecane | 0.25 | 0.4 | 0.5 |
| | polysorbate-80 | 0.2 | 0.32 | 0.4 |
| D | caprylyl glycol | 0.5 | 0.5 | 0.5 |
| | soluble collagen | 1.5 | 1.5 | 1.5 |

Colorful skin care gels of Examples 1 to 3 are prepared by the following steps:

(1) wetting sodium hyaluronate in Group A with propylene glycol, then adding them into an aqueous phase pot and stirring, adding other components in Group A successively to form an aqueous phase pot mixture, then heating the mixture to 80, and keeping the temperature constant for 10 minutes;

(2) adding raw materials in Group B successively into an emulsifying pot, stirring and mixing them thoroughly, and then heating them to 70;

(3) mixing components in Group C thoroughly, then adding them into the emulsifying pot, and stirring them until homogenous;

(4) operating the emulsifying pot at a stirring speed of 300 revolutions per minute, and absorbing the components in Group A by vacuum suction, maintaining vacuum during reaction, after homogenizing for 5 minutes, obtaining an emulsifying pot mixture;

(5) cooling the emulsifying pot mixture to below 45 by stirring, adding components in Group D to get a final mixture, after thorough stirring and homogenizing, cooling the final mixture to 38 and discharging it; and

(6) passing inspection, discharging and filling in bottles.

Appearance of the product is transparent, and lavender.

Examples of Testing Effect:

1. Moisture-retention property test: according to method in QB/T 4256-2011 Guideline for Evaluation of Cosmetics Moisturization Efficacy, efficacy test for 8-hour moisturization on human is carried out by using colorful gels in Examples 1 to 3, and testing results are shown as follows:

| | Test Point | P value | Conclusion |
|---|---|---|---|
| Test for water contents of | T0_{applied area}&T0_{control area} | 0.7588* | Water contents of cuticle in applied |
| cuticle in applied area and | | | area and control area before |
| control area before | | | applying sample are the same |
| treating with sample | | | |
| Test for difference value | Δ (T1-T0) T0_{applied area}& | <0.0001** | The increase of water content of |
| between the initial value | Δ (T1-T0)_{control area} | | cuticle in applied area after being |
| and water content of | | | applied with samples for 1 hour has |
| cuticle in applied area and | | | a significant difference from |
| control area at each time | | | control area |
| point | Δ (T2-T0) T0_{applied area}& | <0.0001** | The increase of water content of |
| | Δ (T2-T0)_{control area} | | cuticle in applied area after being |
| | | | applied with samples for 4 hours |
| | | | has a significant difference from |
| | | | control area |
| | Δ (T3-T0) T0_{applied area}& | <0.0001** | The increase of water content of |
| | Δ (T3-T0)_{control area} | | cuticle in applied area after being |
| | | | applied with samples for 8 hours |
| | | | has a significant difference from |
| | | | control area |

Note:
statistical method:
*single-factor analysis of variance;
**independent sample t-test, there is a significant difference when P value < 0.05.

2. Irritation Evaluation (Human Skin Patch Test Result)

(1) Test sample: colorful gel cosmetic finished product of Examples 1 to 3 according to the present invention.

(2) Negative control: blank control.

(3) Subjects: 25 persons in total, 12 males and 13 females, aged 18˜30, meeting the entry criteria of volunteer.

(4) Method of patch test: enclosed patch test is used; qualified patch test apparatus is selected, about 0.02 ml˜0.025 ml test sample is applied on the inner side of the spot test apparatus, and the patch test apparatus adheres to the flexor side of forearm of the subject with tape for external use, the test sample is removed after 24 hours, and 30 minutes after the spot test apparatus applying test sample is removed, skin reaction is observed after impression disappears, and results are recorded according to skin reaction grading criteria in Hygienic Standard for Cosmetics, 2007 edition.

(5) Results evaluation: reaction results are recorded according to the following table (skin adverse reaction grading criteria).

| Reaction | | |
|---|---|---|
| degree | Grade | Skin reaction |
| + | 0 | Negative reaction |
| ± | 1 | Suspected reaction; only weak spots |
| + | 2 | Weak positive reaction (erythema reaction); |
| | | erythema, infiltration, edema, possible papule |
| ++ | 3 | Strong positive reaction (herpes reaction); erythema, |
| | | infiltration, edema, possible papule; the reaction may |
| | | spread beyond the test area |
| +++ | 4 | Extremely strong positive reaction (confluent herpes |
| | | reaction); obvious erythema, severe infiltration, |
| | | edema, confluent herpes; the reaction spreads beyond |
| | | the test area |

(6) Test results are shown in the following table:

| | | | Number of people with different skin reactions in patch test | | | | |
|---|---|---|---|---|---|---|---|
| | | | Grade 0 | Grade 1 | Grade 2 | Grade 3 | Grade 4 |
| | Number of | Observing | (number of | (number of | (number of | (number of | (number of |
| Group | Subjects | time | examples) | examples) | examples) | examples) | examples) |
| Example 1 | 25 | 24 h | 25 | 0 | 0 | 0 | 0 |
| Example 2 | 25 | 24 h | 25 | 0 | 0 | 0 | 0 |
| Example 3 | 25 | 24 h | 25 | 0 | 0 | 0 | 0 |
| Control | 25 | 24 h | 23 | 2 | 0 | 0 | 0 |

The above results show that 25 subjects all have negative reaction on they skin (grading: Grade 0) in Examples 1, 2, and 3. According to skin reaction grading criteria in Hygienic Standard for Cosmetics, 2007 edition, the samples have no skin adverse effect on human skin.

As can be seen from the above test results, the colorful skin care gel of Examples 1 to 3 according to the present invention has transparent appearance, and it is beautiful and fashionable due to its lavender color by adjusting refractive indexes of aqueous phase and oil phase; it also contains a great amount of nourishing grease, making the combination of both long-acting and short-acting double-effect ultra moisturizing components possible, and has an excellent moisturizing performance; and moreover, the present invention applies special thickening and emulsifying system, the obtained product has smooth texture, and it is without preservatives, safe and environmentally friendly.

What is claimed is:

1. A colorful gel, comprising following components by weight percent:
20.0% to 25.0% by weight of glycerin,
20.0% to 25.0% by weight of propylene glycol,
1.0% by weight of sea algae extract,
05% by weight of disodium EDTA,
0.06% by weight of allantoin,
0.05% by weight of sodium hyaluronate,
20.0% to 30.0% by weight of cyclopentasiloxane,
5.0% to 10.0% by weight of dimethicone,
5% to 1.0% by weight of sodium acrylate/sodium acryloyldimethyl taurate copolymer,
0.25% to 0.5% by weight of isohexadecane,
0.2% to 0.4% by weight of polysorbate-80,
1.5% by weight of soluble collagen,
0.5% by weight of caprylyl glycol, and water as the rest.

2. A method for preparing colorful gel of claim 1, comprising the steps of:
(1) wetting sodium hyaluronate with propylene glycol, then adding them into an aqueous phase pot and stirring, and adding deionized water, glycerin, sea algae extract, disodium EDTA, and allantoin successively to form an aqueous phase pot mixture, then heating the mixture to 80, and keeping the temperature constant for 10 minutes;
(2) adding dimethicone and part of cyclopentasiloxane successively into an emulsifying pot, stirring and mixing them thoroughly, and then heating them to 70° C.;
(3) mixing remaining cyclopentasiloxane, sodium acrylate/sodium acryloyldimethyl taurate copolymer, isohexadecane and polysorbate-80 thoroughly, then adding them into the emulsifying pot, and stirring them until homogenous;
(4) operating the emulsifying pot at a stirring speed of 300 revolutions per minute, and absorbing the mixture prepared instep (1) by vacuum suction, maintaining vacuum during reaction, after homogenizing for 5 minutes, obtaining an emulsifying pot mixture; and
(5) cooling the emulsifying pot mixture to below 45° C. by stirring, adding caprylyl glycol and soluble collagen to get a final mixture, after thorough stirring and homogenizing, cooling the final mixture to 38, and discharging it.

3. The method of claim 2, wherein the cyclopentasiloxane used in step (2) accounts for 95˜97% of all the cyclopentasiloxane by weight.
